(19) United States
(12) Patent Application Publication
(10) Pub. No.: US 2014/0305429 A1
(43) Pub. Date: Oct. 16, 2014

(54) METHOD AND SYSTEM FOR ELECTRONIC MDI MODEL

(71) Applicant: CHIESI FARMACEUTICI S.P.A., PARMA (IT)

(72) Inventor: David Andrew Lewis, Parma (IT)

(21) Appl. No.: 14/359,181

(22) PCT Filed: Dec. 3, 2012
(86) PCT No.: PCT/EP2012/074278

§ 371 Date: May 19, 2014

(30) Foreign Application Priority Data
Dec. 5, 2011 (EP) 11192004.7

(51) Int. Cl. A61M 15/00

(52) U.S. Cl. CPC A61M 15/009; A61M 15/002; A61M 15/0066; A61M 15/0003
USPC 128/200.19; 128/200.23

(57) ABSTRACT

A method and system for optimizing the dispensing of aerosol medicaments by “pulsing” a total dose volume as a series of shorter, low volume bursts is described. Aerosol performance when metering at a low volume e.g. <10 μL is enhanced by an increase in the fine particle fraction, particularly when pulsing a dose to achieve a high total dose volume. By utilising a solenoid valve, the system can deliver a medicament in a single low volume pulse; or in multiple low volume pulses. Performance can be tailored to obtain a preferred fine particle dose and fraction. By manipulating the solenoid valve timings, a single formulation with a concentration X may be used to provide a range of doses. A minimum interval between pulses to achieve separate “non-interacting” plumes which allow keeping total inhalation time comparable to a conventional single dose MDI actuation is also described.

### FIELD OF TECHNOLOGY

The present disclosure relates to pressurized Meter Dose Inhalers (MDI) and more particularly to a device and a method for dispensing aerosol medicament by means of an MDI combined with an electronic valve.

### BACKGROUND

Pressurized metered dose inhalers (pMDIs or simply MDIs) are well known devices for administering pharmaceutical products to the respiratory tract by inhalation. A MDI comprises an actuator in which a pressure resistant aerosol canister or container, typically filled with a medicinal formulation comprising a drug dissolved or in form of micronized drug particles suspended in a liquefied propellant mixture with suitable excipients, is inserted and where the container is fitted with a metering valve. The canister is normally provided with a metering valve comprising a metering chamber, for measuring discrete doses of the medicinal formulation, connected to a hollow valve stem. A typical actuator has a valve stem block, which receives the hollow valve stem of the aerosol canister and a nozzle orifice, having normally a diameter between 0.22 to 0.42 mm, which serves to propel the aerosol towards a mouthpiece opening through which the dose of the aerosol is dispensed to the patient as an inhalable cloud or plume.

Actuation of the metering valve allows a small portion of the spray product to be released whereby the pressure of the liquefied propellant carries the dissolved drug or the suspended micronized drug particles out of the container to the patient

As mentioned above, MDIs use a propellant to expel droplets containing the pharmaceutical product to the respiratory tract as an aerosol. Suitable propellants may be the hydrofluoralkane (HFA) propellants and in particular HFA 134a (1,1,1,2-tetrafluoroethane) and/or HFA 227 (1,1,1,2,3,3,3-heptafluoropropane).

Formulations for aerosol administration via MDIs can be solutions or suspensions. In suspension formulations the micronized particles of the drug are characterised by the log-normal frequency function and consist of particles ranging in size from 1 to 10 micrometers approximately. Suspension type formulations appear satisfactory at the time of preparation but then they may physically degrade during storage. Physical instability of suspensions may be characterised by particle aggregation, crystal growth or a combination of the two, and the result could be a therapeutically ineffective formulation. Solution formulations offer the advantage of being homogeneous, with the active ingredient and excipients completely dissolved in the propellant vehicle comprising a mixture with suitable co-solvents, such as ethanol, or other excipients. Solution formulations also obviate physical stability problems associated with suspension formulations so assuring more consistent uniform dosage administration.

The performance and efficiency of an aerosol device, such as a pMDI, is a function of the dose deposited at the appropriate site in the lungs. Deposition is affected by several factors, of which the most important are the uniformity of delivered dose and the reproducibility and the aerodynamic particle size of the particles in the aerosol cloud. Solid particles and/or droplets in an aerosol formulation can be characterized by their mass median aerodynamic diameter (MMAD).

Respirable particles are generally considered to be those with a MMAD less than 5 μm (in particular <4.7 μm) and the total amount of particles below 5 μm is defined as Fine Particle Dose (FPD). The ratio between the Fine Particle Dose and the Delivered Dose is defined as Fine Particle Fraction (FPF).

With solution formulations it is known that the efficiency of the atomization (expressed as FPD or FPF) is inversely proportional to the fourth root of the atomized volume. Therefore to obtain a highly efficient atomization in terms of FPD and FPF a very low volume of formulation should be atomized. On the other hand, with state of the art mechanical metering valves for medical aerosols, having a volume from 20 to 100 μl it is extremely difficult to dose the aerosol formulation so that a precise small volume is metered. Conventional mechanical metered valves cannot measure a volume lower than or equal to 20 μl and provide acceptable dose reproducibility.

Also, either a decrease of HFA content or a reduction in the volume of the valve is detrimental to the amount of drug that is possible to solubilize. The challenge therefore becomes having the possibility to atomize with high efficiency large volumes (i.e. 100 μl or more) and/or formulations containing lower amounts of HFA. It is known to combine electronic components (e.g. a solenoid valve) to traditional MDIs in order to obtain a more precise dosage with lower volumes. Patent EP 0111163 for example describes a device with an electronic component for metering a predetermined dose of medicament. There are two main components: the solenoid valve which opens and closes according to the electronic timing arrangement and the diffuser element, an oscillating system designed to atomise the inhalation fluid released by the opening of the valve. The pressure for driving the fluid from the container is from a spring-mounted piston, applying a force to the inhalation liquid. The duration of the valve opening determines the amount of dose released. However the device used is not a typical pressurised metered dose inhaler for a medicinal formulation because the pressure for driving the fluid from the container is from a spring-mounted piston, applying a force to the inhalation liquid.

Patent application WO 87/04354 describes a system where a solenoid valve is used to meter a dose of a conventional MDI. The MDI is held in the actuated position and the dose is released upon valve opening in response to an electronic or mechanical signal. The volume of dose is programmable according to the mass discharge. The valve may be pulsed open and closed to achieve a total dose volume over multiple short bursts. Even if it is said that this approach enhances efficiency and improves drug delivery no practical example or demonstration of this approach has been provided.

An improved electronic assisted MDI capable of dispensing an optimal dosage of aerosol formulation so that atomisation is performed to yield an inhaled medicament would be greatly appreciated.

### OBJECTS OF THE DISCLOSURE

It is an object of the present disclosure to improve performances or overcome at least some of the problems associated with the prior art.

### SUMMARY

The present disclosure provides a method and system as set out in the accompanying claims.

According to one aspect of the present disclosure there is provided a method for generating an aerosol cloud containing a high fine particle dose of a medicament with a device including a pressurized Meter Dose Inhaler (MDI) reservoir containing a solution formulation of a medicament operated with HFA propellants, the MDI reservoir being connected to an electronic valve, the valve being adapted for receiving control signals from a microprocessor, the method including the steps of: maintaining in a storage memory at least one set of medicament parameters, each set of parameters including a measure indicative of a total amount of aerosol medicament to be dispensed during a medicament session; the microprocessor controlling the opening of the electronic valve, allowing the total amount of aerosol medicament being dispensed during a total inhalation time with a plurality of successive low volume pulses, the time interval between successive low volume pulses being less than 100 ms and the volume of medicament delivered during each single pulse being less than 5 μl so that the total inhalation time is minimized, while allowing the predetermined total amount of aerosol medicament being delivered.

In a preferred embodiment of the present invention the duration of each low volume pulse is determined so that the fine particle fraction (FPF) of the aerosol medicament is maximized and the amount of FPF of aerosol delivered during each single pulse is calculated according to the following formula:

$$FPF ( \% ) = \frac{k}{{V}_{{P}^{0.25}}}$$

Where the scaling factor k is dependant upon the HFA content of the system and nozzle characteristics (Lewis, D. A. et al (2004) ‘Theory and Practice with Solution Systems’ Proc. Respiratory Drug Delivery IX, Vol 1, 109-115).

In a second aspect of the invention the time interval between successive low volume pulses is determined so as to maximize fine particle fraction in atomizing a high volume formulation.

In a preferred embodiment the time interval between successive low volume pulses is 50 ms and the volume of medicament delivered during each single pulse is 2 μ.

In a further aspect of the invention the storage memory includes a plurality of sets of medicament parameters and the calculation of the time interval between the plurality of successive low volume pulses and the amount of medicament delivered during each single pulse is performed responsive to a user selection of one of the plurality of sets of medicament parameters.

According to a preferred embodiment of the invention, the HFA propellants include e.g. HFA 134a (1,1,1,2-tetrafluoroethane), HFA 227 (1,1,1,2,3,3,3-heptafluoroproane) or a mixture thereof.

In an embodiment of the invention, the MDI is connected to a plurality of electronic valve and to a plurality of reservoirs, each reservoir being coupled to at least one of the plurality of electronic valve, each valve being adapted to deliver a different aerosol formulation.

In yet another aspect of the present invention we provide a system which includes components adapted to implement the above method.

Also a device is disclosed for dispensing an aerosol medicament, the device including: a pressurized Meter Dose Inhaler (MDI) operated with HFA propellants; at least one reservoir adapted to contain aerosol medicaments; at least one electronic valve, being connected to the MDI; a microprocessor for controlling the opening of the electronic valve, allowing a predetermined amount of aerosol medicament being dispensed during a total inhalation time with a plurality of successive low volume pulses, the time interval between successive low volume pulses being adjusted so that the total inhalation time is minimized, while allowing the predetermined amount of aerosol medicament being delivered.

In a further embodiment the device includes a plurality (e.g. 2) of electronic valves and a plurality of reservoirs, each reservoir being coupled to at least one of the plurality of electronic valves, each valve being adapted to deliver a different aerosol formulation.

A still further aspect of the present invention provides a computer program for performing the above described method

The method and system according to preferred embodiments of the present invention allows optimizing the dispensing of aerosol medicaments by “pulsing” a total dose volume as a series of shorter, low volume bursts. The interval between two pulses is reduced as much as possible in order not to have interacting plumes. Aerosol performance when metering at a low volume e.g. <10 μL is enhanced by an increase in the fine particle fraction, particularly when pulsing a dose to achieve a high total dose volume. By utilising a solenoid valve, we can deliver a medicament in a single low volume pulse; or in multiple low volume pulses. Performance can be tailored to obtain a preferred fine particle dose and fraction. By manipulating the solenoid valve timings, a single formulation with a concentration X may be used to provide a range of doses e.g. 50 μg; 100 μg; 200 μg; 400 μg. We have explored the minimum interval between pulses to achieve separate “non-interacting” plumes which allow keeping total inhalation time comparable to a conventional single dose MDI actuation. Furthermore, the flexibility of this system allows exploring multiple valve systems with separate control to synchronise alternate dosing from two or more separate formulations whilst achieving improved, but individual, aerosol characteristics.

### BRIEF DESCRIPTION OF THE DRAWINGS

Reference will now be made, by way of example, to the accompanying drawings, in which:

FIG. 1 is a schematic diagram of An Electronic MDI Model (EMM) according to a preferred embodiment of the present invention;

FIG. 2 shows a diagram of time gap between pulses according to an embodiment of the present invention;

FIGS. 3a and 3b represent respectively a single and a dual EMM system according to an embodiment of the present invention;

FIGS. 4-7 show diagrams of various parameters of a fine particle dispensing method according to an embodiment of the present invention;

FIGS. 8, 9A and 9B show views of sample actuators 1 and 2e connected to the respective micro-dispensing nozzle valve;

FIGS. 10 and 11 show the effect of pulse separation on drug delivery and on the delivery efficiency of formulation E from sample actuators 1 and 2e.

### DETAILED DESCRIPTION OF PREFERRED EMBODIMENTS

The method according to a preferred embodiment of the present invention uses solenoid valves to meter a dose from a conventional MDI. A propellant-based formulation is used to impart the pressure driving atomisation. The electronic solenoid valve used in a preferred embodiment to model a conventional MDI can operate up to 8 bar; suitable for traditional HFA propellants, e.g. HFA 134a (1,1,1,2-tetrafluoroethane), HFA 227 (1,1,1,2,3,3,3-heptafluoroproane) or a mixture thereof. The application of the electronic signal to the valve determines the duration the valve is open; which subsequently determines dose volume. By applying multiple signals over time the dose may be effectively “pulsed” to achieve a total dose volume. By pulsing small volumes, an increase in the efficiency of the aerosolised dose may be achieved; enhancing drug delivery.

The efficacy of an MDI device is a function of the dose deposited at the appropriate site in the lungs. Deposition is affected by the aerodynamic particle size distribution of the formulation which may be characterised in vitro through several parameters.

The aerodynamic particle size distribution of the formulation of the invention may be characterized using a Cascade Impactor according to the procedure described in the European Pharmacopoeia 6^{th} edition, 2009 (6.5), part 2.09.18. An Apparatus E, operating at a flow rate range of 30 l/min to 100 l/min or an Apparatus D—Andersen Cascade Impactor (ACI)-, operating at a flow rate of 28.3 l/min. Deposition of the drug on each ACI plate is determined by high performance liquid chromatography (HPLC).

The following parameters of the particles emitted by a pressurized MDI may be determined:
mass median aerodynamic diameter (MMAD) is the diameter around which the mass aerodynamic diameters of the emitted particles are distributed equally;
delivered dose is calculated from the cumulative deposition in the ACI, divided by the number of actuations per experiment;
respirable dose (fine particle dose=FPD) is obtained from the deposition from Stages 3 (S3) to filter (AF) of the ACI, corresponding to particles of diameter ≦4.7 μm, divided by the number of actuations per experiment;
respirable fraction (fine particle fraction=FPF) which is the percent ratio between the respirable dose and the delivered dose;
“superfine” dose is obtained from the deposition from Stages 6 (S6) to filter, corresponding to particles of diameter ≦1.1 microns, divided by the number of actuations per experiment;

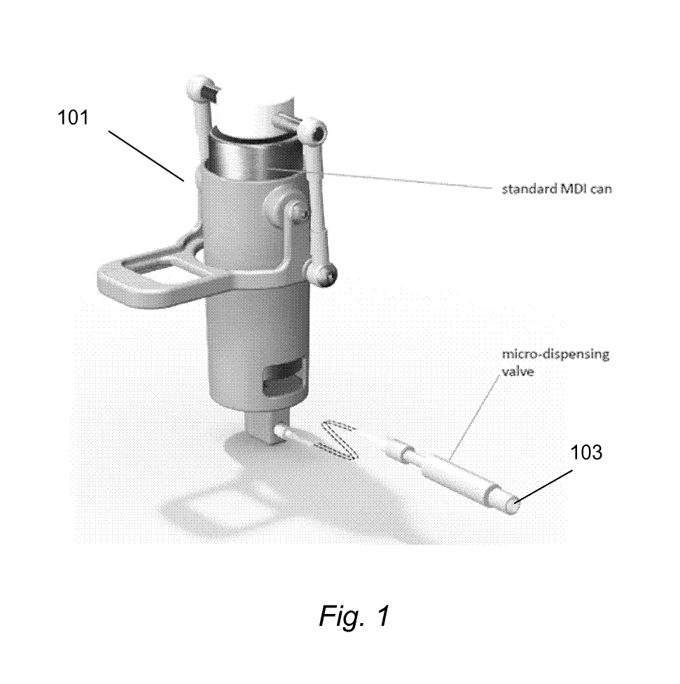

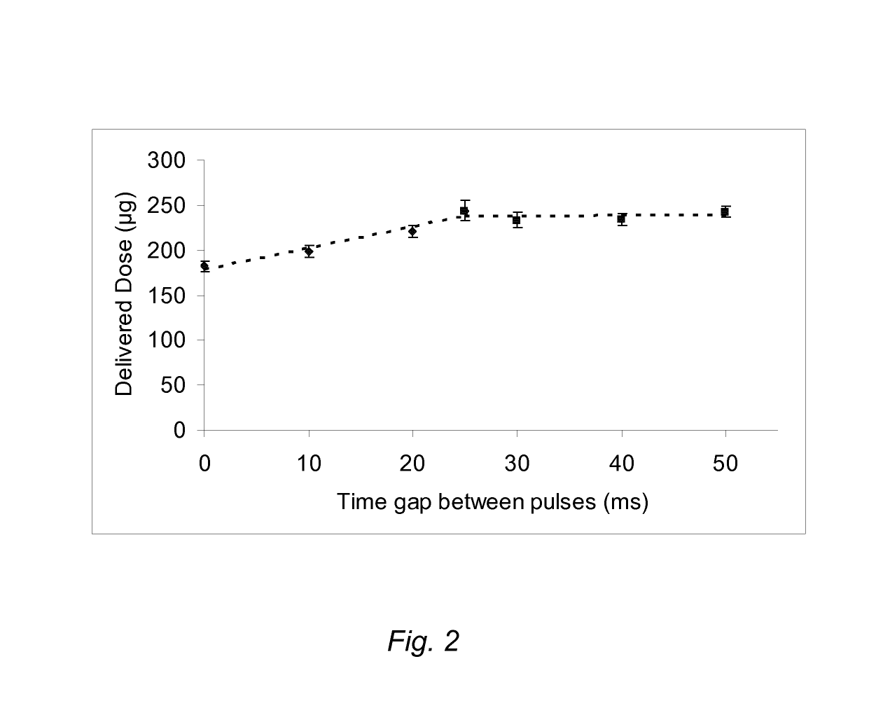

FIG. 1 shows an Electronic MDI Model (EMM) used to implement the method according to a preferred embodiment of the present invention. An MDI valve-canister 101 is connected e.g. by means of a rubber tube to a micro-dispensing valve 103, e.g. a solenoid valve. In a preferred embodiment of the present invention the arrangement allows the MDI valve-can assembly 101 provided with a continuous valve to be held in an actuated position such that a constant supply of liquid formulation is delivered to the micro-dispensing valve (e.g. a solenoid valve) 103 connected to a commercially available nozzle structure suitable to dispense medicinal aerosol pressurised with conventional HFA propellants such as HFA 134a and/or HFA 227. In a preferred embodiment the EMM assembly is connected with a dispenser (not shown) which can be used by the patient for inhalation. The solenoid micro-dispensing valve 103 is normally inserted in a conventional MDI actuator at the level of the stem block, as shown in FIG. 1 or in a suitable designed actuator as shown in FIG. 2 . . . .

The electronically controlled model metered dose inhaler system using a method according to a preferred embodiment of the present invention is able to deliver low volumes, e.g. from 50 μl down to 1-2 μl per pulse. Selection of either the commercially available “tube” or “long” nozzle (both with 0.254 mm internal diameter and 17.78 mm length but differing in the outer diameter of the outlet, 0.51 and 1.27 mm respectively) in combination with a micro-dispensing solenoid valve allows the atomisation performance of conventional 0.30 mm or 0.42 mm nozzle diameter actuators for pressurised MDI to be mimicked. The so called “short” nozzle (with 0.254 mm internal diameter, 8.84 mm length and 2.5 mm outer diameter) may also be used. The versatility of nozzle positioning combined with the ability to control multiple reservoir-nozzle systems allows the flexible construction of novel drug delivery systems that can be screened for drug delivery advantages. The fine particle fraction of an MDI has previously been found to be dependent upon the inverse fourth root of dose volume (see for example Lewis, D. A. et al (2004). ‘Theory and Practice with Solution Systems’. Proc. Respiratory Drug Delivery IX, Vol 1, 109-115). This report has identified that the fine particle fraction of multiple reservoir-nozzle pulsing systems is dependent upon the inverse fourth root of the total pulse volume, as opposed to the total dose volume. Since it is possible to deliver a total dose volume of 50 μl as a series of low volume pulses within the time required to deliver a single 50 μl dose from a standard 0.30 mm actuator, efficiency may be greatly increased using this pulsing method. A post nozzle break-up system, tube and hole actuators incorporating the EMM system have been found to illicit changes in plumes post-orifice. Reduction in particle size distributions have been related to increases in the velocity of sheath air surrounding the emerging plume. Simultaneous atomizing from two nozzles resulted in plume interaction, mixing of formulation (post nozzle) and distributions from each nozzle becoming similar.

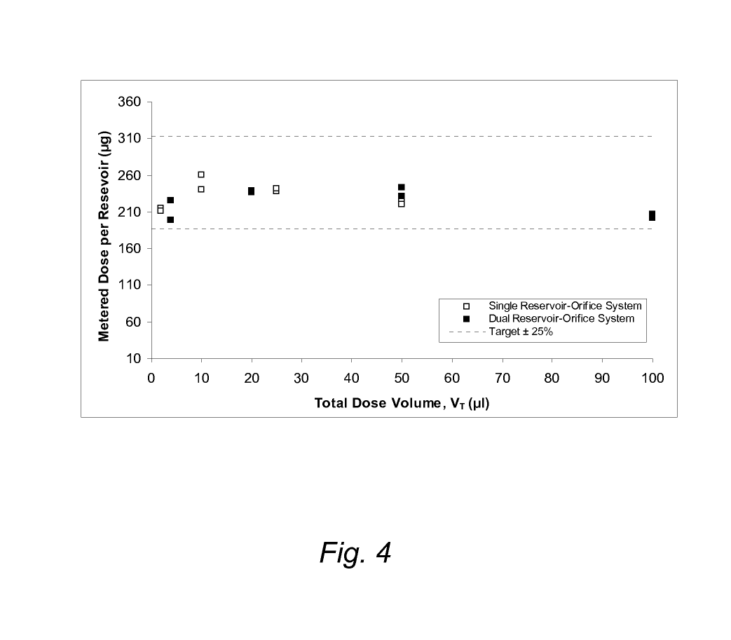

In order to achieve discrete doses of formulation, the time required to separate multiple pulses of formulation must be determined. FIG. 4 presents the delivered doses from 5×10 μl pulses of a beclometasone diipropionate BDP 50 μg/10 μl, 15% w/w ethanol, HFA 134a to 100% w/w formulation with different time intervals separating the pulses.

Sets of pulses separated by more than 25 ms gave consistent delivered doses. There was no overlap between pulses using these programs, with each pulse delivering a separate, discrete dose of formulation.

It is therefore possible to deliver the 5×10 μl pulses using the EMM long 0.254 mm nozzle in ˜0.19 s; shorter than a standard MDI using the equivalent 0.30 mm actuator, which delivers an unbroken 50 μl dose in ˜0.27 s.

This introduces the possibility of delivering two formulations from separate reservoirs, with no interaction between the pulses, within a single inhalation.

The EMM offers the opportunity to pulse doses from either a single or multiple reservoir-nozzle system. This is useful to evaluate if such delivery systems have potential therapeutic advantage. The two test formulations used during this section were:

Formulation A

BDP 250 μg/50 μl (0.44% w/w), 15% w/w ethanol, 84.56% w/w HFA 134a (to 100% w/w).

Formulation B

Budesonide 200 μg/50 μl (0.35% w/w), 15% w/w ethanol, 1.3% w/w glycerol, 83.35% w/w HFA 134a (to 100% w/w).

In the formulations above the % w/w means the amount by weight of the component, expressed as percent with respect to the total weight of the composition.

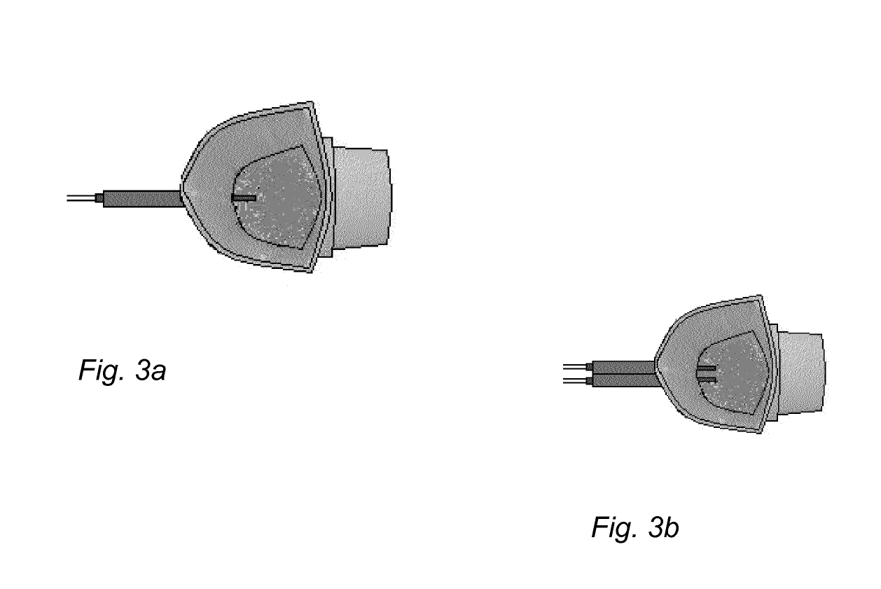

In this section data from four delivery modes are described; these were:
1) A single EMM (long nozzle) system (see FIG. 3A) that delivers five 10 μl pulses from Formulation A or five 10 μl pulses from Formulation B, i.e. A, A, A, A, A or B, B, B, B, B.
2) A dual EMM (long nozzle) system (see FIG. 3B) that delivers a 10 μl pulse from Formulation A followed by a 10 μl pulse from Formulation B; repeated such that a total of 5 doses are fired from each EMM, i.e. A, B, A, B, A, B, A, B, A, B.
3) A dual EMM (long nozzle) system (see FIG. 3B) that delivers five 10 μl pulses from Formulation A followed by five 10 μl pulses from Formulation B, i.e. A, A, A, A, A, B, B, B, B, B.
4) A dual EMM (long nozzle) system (see FIG. 3B) that delivers a 10 μl pulse from

Formulation A at the same time as delivering a 10p1 pulse from Formulation B; repeated such that a that a total of 5 doses are fired from each EMM, i.e. A&B, A&B, A&B, A&B, A&B.

The data collected using the four delivery modes is presented in Table 1. The delivered dose is reduced with the dual reservoir-nozzle systems compared to that of the single reservoir-nozzle systems. It is proposed that this reduction may be due to the affects of orientation and positioning of multiple nozzles, and these variables are currently under investigation.

TABLE 1
Program for Dual Micro-Dispensing Valves (Excel Format)

| | EMM Delivering Budesonide | | | | EMM Delivering BDP | | | |
|---|---|---|---|---|---|---|---|---|
| | 200 μg | | | | 250 μg | | | |
| | Single or Dual System: | | | | | | | |
| | Single | Dual | Dual | Dual | Single | Dual | Dual | Dual |
| | Delivery Mode: | | | | | | | |
| | | 2 | 3 | 4 | 1 | 2 | 3 | 4 |
| | 1 | (Alt.) | (Sep.) | (Sim.) | (−) | (Alt.) | (Sep.) | (Sim.) |
| Metered Dose (μg) | 197 | 165 | 165 | 195 | 251 | 241 | 234 | 245 |
| Delivered Dose (μg) | 185 | 141 | 144 | 157 | 235 | 207 | 197 | 189 |
| FPD (μg) | 45 | 40 | 37 | 28 | 68 | 66 | 57 | 35 |
| FPF (μg) | 24 | 29 | 26 | 18 | 29 | 32 | 29 | 19 |
| MMAD (μm) | 3.4 | 3.1 | 3.2 | 3.1 | 2.1 | 2.0 | 2.1 | 2.2 |

The fine particle fraction of the single reservoir-nozzle system and the dual reservoir-nozzle systems with alternating pulses (delivery mode 2) or separated pulses (delivery mode 3) are comparable (BDP 24-29% and Budesonide 29-32%). However, there is a significant drop in the fine particle fraction when simultaneous pulsing (delivery mode 4) is used (FPF of BDP delivery=18% and Budesonide=19%). The reason for this is explained in the following section.

Pulse Volume and Total Dose Volume

The fine particle fraction of a metered dose inhaler has previously been published to be dependent upon the inverse fourth root of dose volume (Lewis D. A. et al, 2004). This section demonstrates that the fine particle fraction of multiple reservoir-nozzle pulsing systems is dependent upon the inverse fourth root of the total pulse volume.

Table 2 presents eight BDP HFA 134a systems investigated. Systems were either single reservoir or dual reservoir; each reservoir containing an MDI from the same batch of Formulation A (0.44% w/w BDP, 15% w/w ethanol and 84.56% w/w HFA 134a). All reservoirs were programmed to meter a total dose volume (V_{T}) of 50 μl.

Four single reservoir systems were investigated: 25×2 μl pulses, 5×10 μl pulses, 2×25 μl pulses and 1×50 μl pulses. The total dose mass for each single reservoir systems was 50.7±3.3 mg.

Likewise, four dual reservoir systems were investigated with parallel, centrally positioned nozzles such that each system mimicked the four single reservoir systems; with two synchronised pulsing reservoirs. The total dose mass of each dual reservoir system was 100.9±8.5 mg (50.4±4.7 mg per reservoir). The Mean metered dose per reservoir for all systems was 227±17 μg; individual values are presented in FIG. 4.

TABLE 2
Eight BDP 250 μg HFA 134a Systems containing 15% w/w Ethanol

| | Pulse Volume (μl), 30 ms Separation | | | | | | | |
|---|---|---|---|---|---|---|---|---|
| Reservoir-Nozzle 1 | 2 | 10 | 25 | 50 | 2 | 10 | 25 | 50 |
| Reservoir-Nozzle 2 | — | — | — | — | 2 | 10 | 25 | 50 |
| Total Pulse Volume, Vₚ | 2 | 10 | 25 | 50 | 4 | 20 | 50 | 100 |
| | Dose Volume (μl) | | | | | | | |
| Number of pulses | 25 | 5 | 2 | 1 | 25 | 5 | 2 | 1 |
| Total Dose Volume, V_{T} | 50 | 50 | 50 | 50 | 100 | 100 | 100 | 100 |

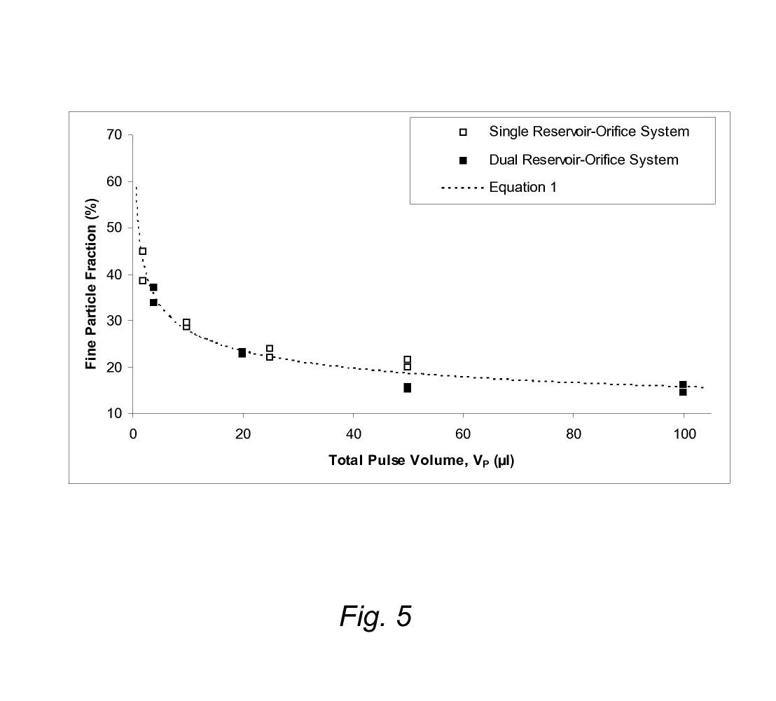

The efficiency of each system was found to be proportional to the inverse fourth root of the total pulse volume, Vₚ, (see FIG. 5). The pulse volume modulates the emitted dose such that the efficiency of delivery from the 50 μl dose (single reservoir systems) or 100 μl dose (dual reservoir systems) is varied between 14 and 45%. The equation for predicting the fine particle fraction of the systems is:

$$FPF ( \% ) = \frac{k}{{V}_{{P}^{0.25}}} \qquad ( 1 )$$

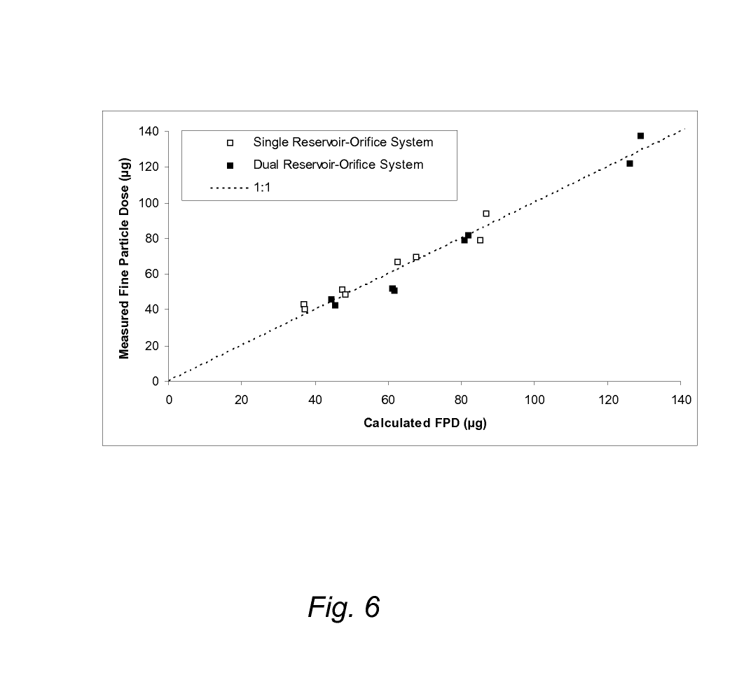

The scaling factor k is dependent upon the HFA content of the system and nozzle characteristics (Lewis D. A. et al, 2004). In the present example the scaling factor k is 49.4 and corresponds to the following formulation A (0.44% w/w BDP; 15% w/w ethanol; and 84.56% w/w HFA 134a) delivered through the “long” nozzle having a 0.254 mm diameter, mounted within a conventional pMDI actuator. The 1:1 relationship between the measured and the calculated FPD is presented in FIG. 6.

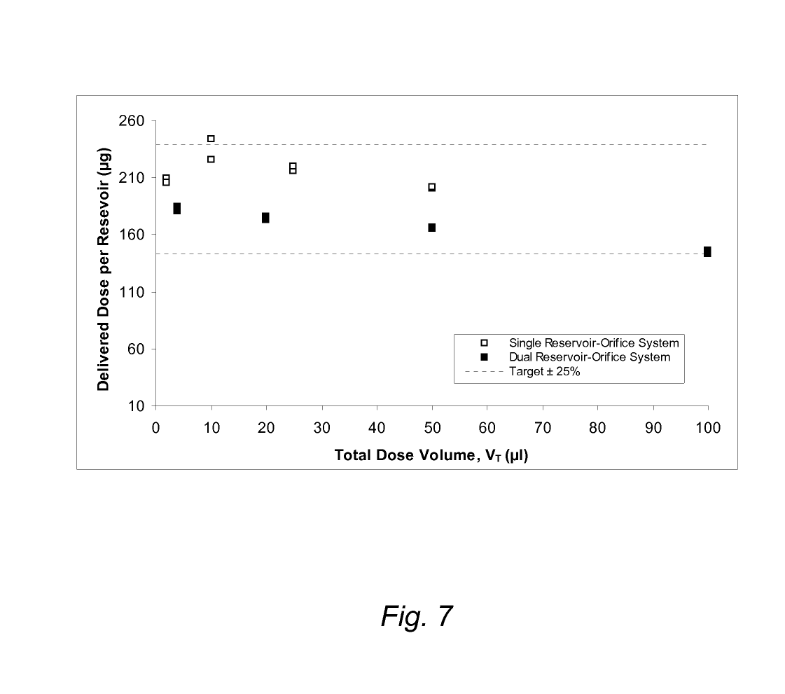

Equation 1 and FIG. 6 demonstrate that it is possible to predict the FPD from HFA 134a systems with a known delivered dose. The complexities of plume interaction with the actuator housing are not currently understood, but the positioning and orientation of the nozzle(s) is known to be important. FIG. 7 highlights that the delivered dose is reduced with the dual reservoir-nozzle system compared to the single reservoir-nozzle system.

Dose Pulsing and Pulse Separation

We have explored the minimum interval between pulses to achieve separate “non-interacting” plumes which allow keeping total inhalation time comparable to a conventional single dose MDI actuation.

A delay between each electrical pulse supplied to the micro-dispensing valve was used to achieve discrete consecutive dosing of the formulation. To evaluate the period of separation between consecutive dispensed doses, the plume duration of each dispensation was measured using audio duration data obtained by a microphone, positioned into a fix position in the vicinity of the MDI. The microphone was connected to a computer and the audio signals of the different measurements were recorded and managed using a specific software through which each trace for each dispensation was selected, zoomed into the beginning and end, cut to leave only the plume duration trace and aligned with the other, analysed and compared.

For each nozzle and formulation tested, audio durations data were determined for target dose volumes of 2, 5, 10, 50 and 100 μl for both:

Formulation C

BDP 50 μg/50 μl (0.087% w/w), 12% w/w ethanol, 87.913% w/w HFA 134a (to 100% w/w), and

Formulation D

100% w/w HFA 134a packaged within equivalent MDI hardware.

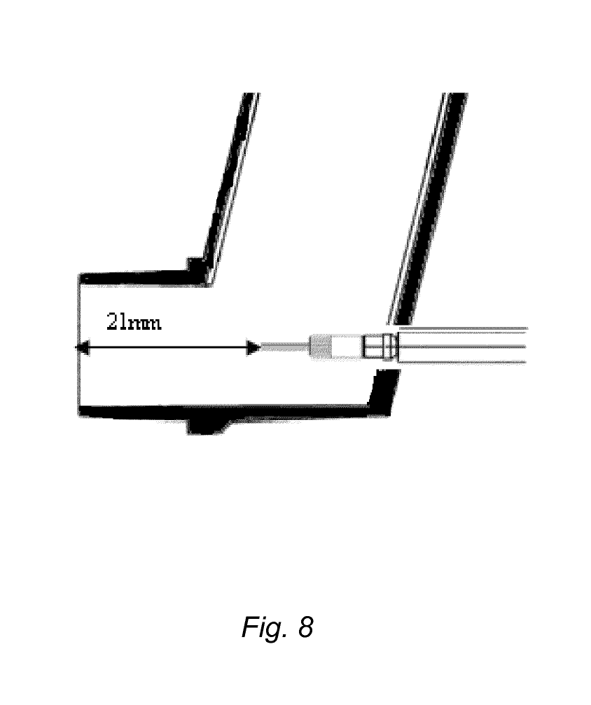

The formulations have been dispensed through the sample 1 actuator of FIG. 8, manufactured by modifying a conventional MDI actuator by removing the stem block, accommodating the micro-dispensing valve through a hole provided into the actuator's back and positioning the nozzle 21 mm from the mouthpiece opening.

All drug data are an average of two consecutive doses sampled from the micro-dispensing valve and fired with an interval of at least 1 minute.

The plume duration, P′, of doses (target volumes: 2, 5, 10, 50 and 100 μl) emitted from the commercially available short, long and tube nozzles are presented in Tables 3, 4 and 5 respectively.

TABLE 3
Audio Plume Duration (n = 5): Short Nozzle

| | Target | Mean Shot | Pulse | Plume | | |
|---|---|---|---|---|---|---|
| | Dose | Weight | Length, P | Duration, P′ | Δt = P′-P | |
| Formulation | (μl) | (mg) | (ms) | (ms) | (ms) | |
| C | 2 | 2.1 | 3 | 30 | 27 | 27 |
| | 5 | 5.4 | 7 | 50 | 43 | 45 ± 2 |
| | 10 | 11.2 | 23 | 67 | 44 | |
| | 50 | 59.2 | 165 | 210 | 45 | |
| | 100 | 109.4 | 332 | 379 | 47 | |
| D | 2 | 2.3 | 2 | 29 | 27 | 27 |
| (100% HFA | 5 | 6.0 | 8 | 51 | 43 | 46 ± 2 |
| 134a) | 10 | 12.2 | 25 | 74 | 49 | |
| | 50 | 62.4 | 170 | 216 | 46 | |
| | 100 | 120.0 | 300 | 345 | 45 | |

TABLE 4
Audio Plume Duration (n = 5): Long Nozzle

| | Target | Mean Shot | Pulse | Plume | | |
|---|---|---|---|---|---|---|
| | Dose | Weight | Length, P | Duration, P′ | Δt = P′-P | |
| Formulation | (μl) | (mg) | (ms) | (ms) | (ms) | |
| C | 2 | 2.3 | 2 | 23 | 21 | 21 |
| | 5 | 6.0 | 7 | 39 | 32 | 40 ± 5 |
| | 10 | 11.6 | 15 | 57 | 42 | |
| | 50 | 59.5 | 94 | 135 | 41 | |
| | 100 | 112.5 | 186 | 230 | 44 | |
| D | 2 | 2.5 | 2 | 31 | 29 | 29 |
| (100% HFA | 5 | 5.8 | 5 | 49 | 44 | 47 ± 3 |
| 134a) | 10 | 12.1 | 13 | 63 | 50 | |
| | 50 | 58.6 | 79 | 125 | 46 | |
| | 100 | 110.7 | 160 | 207 | 47 | |

TABLE 5
Audio Plume Duration (n = 5): Tube Nozzle

| | Target | Mean Shot | Pulse | Plume | | |
|---|---|---|---|---|---|---|
| | Dose | Weight | Length, P | Duration, P′ | Δt = P′-P | |
| Formulation | (μl) | (mg) | (ms) | (ms) | (ms) | |
| C | 2 | 2.2 | 4 | 21 | 17 | 17 |
| | 5 | 6.4 | 15 | 38 | 23 | 26 ± 2 |
| | 10 | 11.6 | 30 | 55 | 25 | |
| | 50 | 60.7 | 170 | 197 | 27 | |
| | 100 | 105.0 | 320 | 347 | 27 | |
| D | 2 | 2.2 | 2 | 32 | 30 | 30 |
| (100% HFA | 5 | 6.3 | 8 | 44 | 36 | 31 ± 4 |
| 134a) | 10 | 11.8 | 19 | 47 | 28 | |
| | 50 | 62.0 | 130 | 160 | 30 | |
| | 100 | 122.4 | 260 | 289 | 29 | |

Shot weight values confirm that increasing the electrical pulse length, P, which is the time that voltage is supplied to the micro-dispensing valve, increases the mass discharged from the nozzle. The length of time the plume was still audible after the completion of the electrical pulse, Δt, was determined by subtracting P values from P′ values. The value of Δt gives the minimum duration that each pulse length, P, should be separated to ensure consecutively emitted plumes are distinct. For the 2 μl target volumes, Δt ranged between 17-27 ms, whilst Δt ranged between 23 and 50 for the 5-100 μl target volumes. Pooling all data gave Δt=36±10 ms.

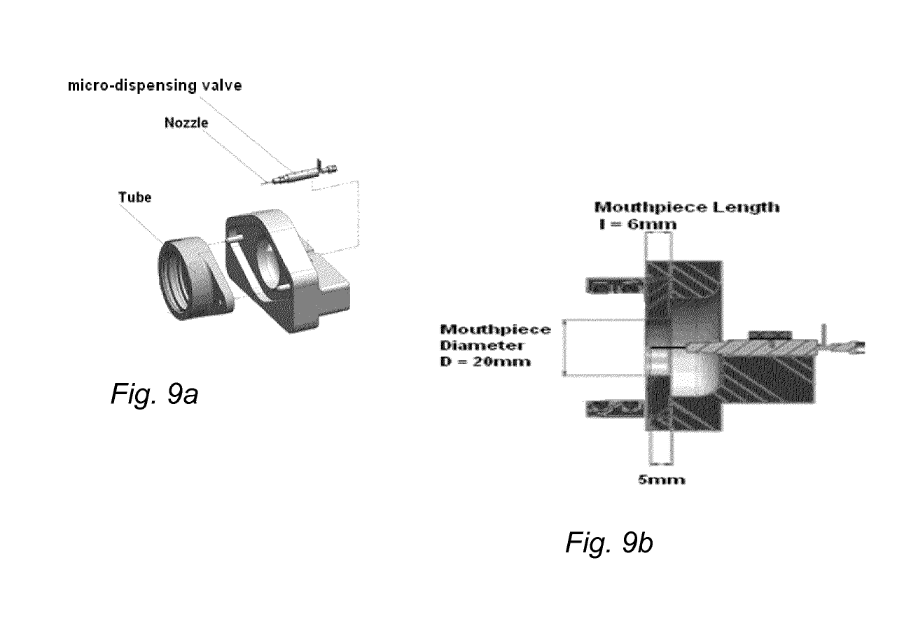

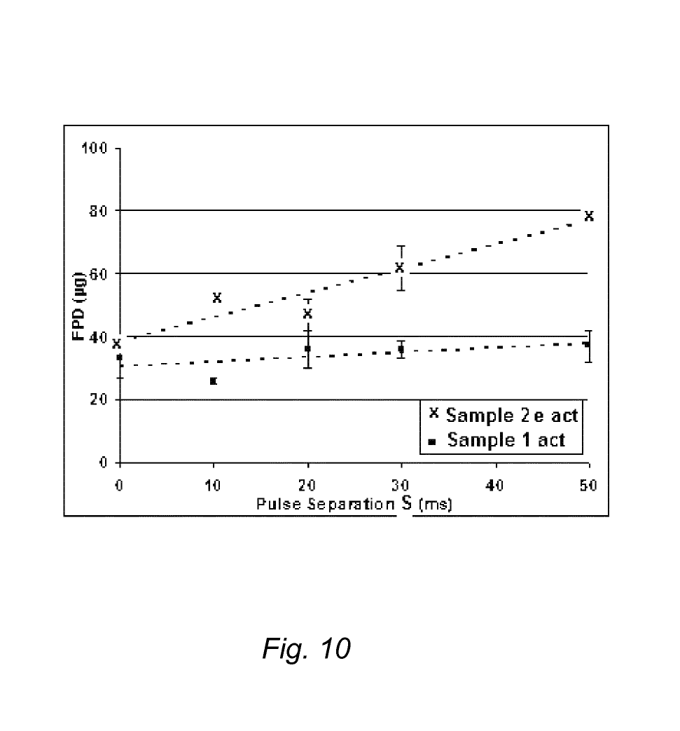

The effect of changing pulse separation, S, between twenty five consecutive 40 ms pulses (approximately 25×2 μl=50 μl) for the administration of Formulation E constituted by 0.17% w/w BDP (100 μg/50 μl), 12% w/w Ethanol, 87.83% w/w HFA 134a is shown in Table 6 when delivered through the sample 1 actuator of FIG. 8 provided with a micro-dispensing valve with the “tube” nozzle, and in Table 7 when delivered through the sample 2e actuator of FIGS. 9A and 9B provided with a micro-dispensing valve with the “tube” nozzle. The results are then graphically compared in FIG. 10. The fine particle dose (FPD) delivery increases linearly for sample 2e actuator of FIGS. 9A and 9B as pulse separation increases (up to a maximum of 78±2 μg). When the delivered dose is pulsed using sample 1 actuator of FIG. 8, little influence is observed on drug delivery performance as pulse separation increases. The data in FIG. 10 demonstrates that drug delivery efficiency can be increased by splitting the metered dose into discrete pulses; however, pulse separation and actuator geometry are highly influential.

TABLE 6
Sample 1 Actuator: Effect of pulse separation upon drug delivery; 25
consecutive 2 μl pulses of Formulation E (BDP target 100 μg/50 μl)

| | Pulse Separation, S (ms) | | | | |
|---|---|---|---|---|---|
| | 0 | 10 | 20 | 30 | 50 |
| Metered | 111 ± 8 | 109 ± 1 | 122 ± 6 | 124 ± 3 | 117 ± 3 |
| Dose (μg) | | | | | |
| Delivered | 108 ± 9 | 106 ± 1 | 117 ± 6 | 120 ± 3 | 114 ± 3 |
| Dose (μg) | | | | | |
| FPD (μg) | 33 ± 6 | 26 ± 1 | 36 ± 6 | 36 ± 3 | 37 ± 5 |
| FPF (%) | 30 ± 5 | 25 ± 1 | 31 ± 4 | 30 ± 3 | 33 ± 5 |
| MMAD | 1.9 ± 0.4 | 1.7 ± 0.1 | 1.9 ± 0.1 | 1.8 ± 0.1 | 2.0 ± 0.1 |
| (μm) | | | | | |
| n | 4 | 3 | 3 | 3 | 3 |

TABLE 7
Sample 2e Actuator: Effect of pulse separation upon drug delivery; 25
consecutive 2 μl pulses of Formulation E (BDP target 100 μg/50 μl)

| | Pulse Separation, S (ms) | | | | |
|---|---|---|---|---|---|
| | 0 | 10 | 20 | 30 | 50 |
| Metered | 123 ± 3 | 108 ± 2 | 127 ± 2 | 112 ± 6 | 130 ± 3 |
| Dose (μg) | | | | | |
| Delivered | 121 ± 3 | 108 ± 2 | 126 ± 2 | 112 ± 6 | 126 ± 3 |
| Dose (μg) | | | | | |
| FPD (μg) | 37 ± 2 | 53 ± 1 | 47 ± 5 | 62 ± 7 | 78 ± 2 |
| FPF (%) | 30 ± 2 | 49 ± 1 | 37 ± 3 | 55 ± 5 | 62 ± 3 |
| MMAD | 1.6 ± 0.1 | 1.5 ± 0.1 | 1.8 ± 0.1 | 1.6 ± 0.1 | 1.8 ± 0.1 |
| (μm) | | | | | |
| n | 3 | 3 | 3 | 3 | 3 |

n = number of replicas of the experiment

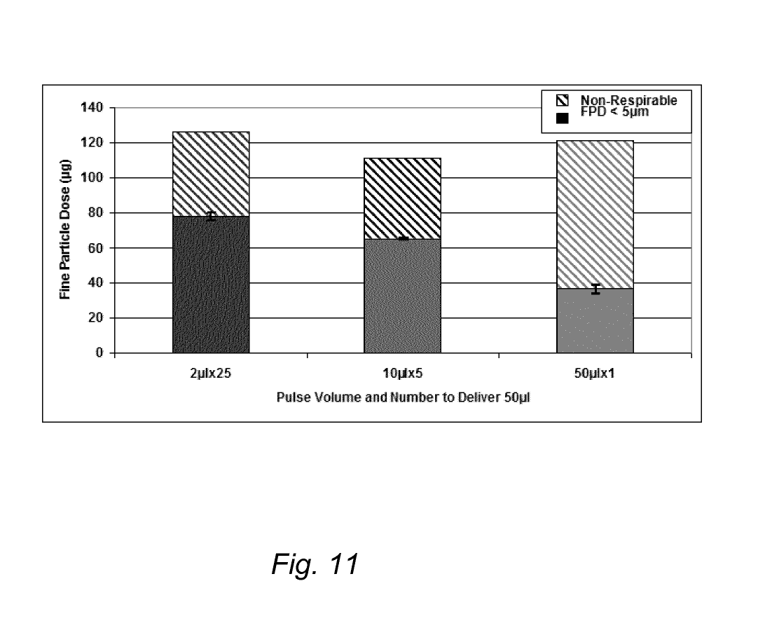

The improved dosing efficiency of sample 2e actuator of FIGS. 9A and 9B was found to diminish as the 50 μl total dose was split into fewer pulses i.e. five 10 μl doses or one 50 μl dose (pulse separation was maintained at 50 ms, see FIG. 11).

In summary, dosing efficiency of HFA formulations can be significantly increased by delivering small pulse volumes (<5 μl, e.g. 2 μl) with a long pulse separation (<100 ms, e.g. 50 ms) and a pertinent selection of actuator housing (e.g. sample actuator 2e).

Length and Diameter of Actuator Mouthpieces

Drug delivery performance of an Electronic MDI Model (EMM) according to the present invention was evaluated for eight alternative sample actuators having two alternative mouthpiece lengths with four different mouthpiece diameters each.

In particular the delivery from sample prototypes series 2 and 3, having mouthpiece length of 6 and 40 mm respectively and mouthpiece diameter of 2, 5, 20 and 35 mm, were determined and compared to the delivery from a conventional actuator housing (sample 1 of FIG. 8).

In all these samples the micro-dispensing valve was fixed centrally within the mouthpiece as shown in FIG. 9B.

A single 20 μl dose of Formulation F, constituted by 0.44% w/w BDP (100 μg/20 μl), 12% ethanol, 87.56% w/w HFA 134a, was delivered by the micro-dispensing valve using a 49 ms pulse.

The results reported in Table 8 show that reducing mouthpiece diameter from 35 mm to 2 mm reduces the mass median aerodynamic diameter (MMAD) from ˜2.0 μm to —0.9 μm at length 40 mm.

The lowest fine particle dose <5 μm (FPD) values observed were 24 μg when the mouthpiece diameter was reduced to 2 mm. Relatively consistent FPD values were observed for mouthpiece diameters 5 mm up to 20 mm (43-47 μg). However, when the mouthpiece diameter was matched to the USP induction port entrance diameter (35 mm) the highest FPD value (57 μg) was observed when the mouthpiece length was 40 mm. The delivered dose appears to be dependent upon both mouthpiece length and diameter. The data in Table 8 demonstrates that mouthpiece geometry (length and diameter) has a significant effect upon the delivered dose, MMAD and FPD.

TABLE 8
ACI data from tube actuators of sample series 2 and 3 compared to
those of sample 1 actuator of FIG. 8

| | | 2 | | | | 3 | | | |
|---|---|---|---|---|---|---|---|---|---|
| Sample actuator series | 1 | a | c | e | g | h | j | l | n |
| Mouthpiece Length (mm) | 21 | 6 | | | | 40 | | | |
| Mouthpiece Diameter (mm) | — | 2 | 5 | 20 | 35 | 2 | 5 | 20 | 35 |
| Metered Dose ( μg) | 97 | 91 | 91 | 97 | 95 | 94 | 98 | 99 | 98 |
| Delivered Dose ( μg) | 91 | 85 | 89 | 96 | 94 | 55 | 59 | 93 | 96 |
| FPD <5 μm ( μg) | 40 | 24 | 47 | 47 | 47 | 24 | 44 | 43 | 57 |
| FPF <5 μm (%) | 43 | 29 | 53 | 49 | 50 | 44 | 75 | 47 | 59 |
| MMAD ( μm) | 1.6 | 1.1 | 1.5 | 2.0 | 1.8 | 0.9 | 1.3 | 1.9 | 1.9 |
| n | 2 | 2 | 2 | 2 | 2 | 2 | 2 | 2 | 2 |

n = number of replicas of the experiment

It will be appreciated that alterations and modifications may be made to the above without departing from the scope of the disclosure. Naturally, in order to satisfy local and specific requirements, a person skilled in the art may apply to the solution described above many modifications and alterations. Particularly, although the present disclosure has been described with a certain degree of particularity with reference to preferred embodiment(s) thereof, it should be understood that various omissions, substitutions and changes in the form and details as well as other embodiments are possible; moreover, it is expressly intended that specific elements and/or method steps described in connection with any disclosed embodiment of the disclosure may be incorporated in any other embodiment as a general matter of design choice.

For example, similar considerations apply if the components (e.g. microprocessor or computers) have different structure or include equivalent units; in any case, it is possible to replace the computers with any code execution entity (such as a PDA, a mobile phone, and the like).

Similar considerations apply if the program (which may be used to implement some embodiments of the disclosure) is structured in a different way, or if additional modules or functions are provided; likewise, the memory structures may be of other types, or may be replaced with equivalent entities (not necessarily consisting of physical storage media). Moreover, the proposed solution lends itself to be implemented with an equivalent method (having similar or additional steps, even in a different order). In any case, the program may take any form suitable to be used by or in connection with any data processing system, such as external or resident software, firmware, or microcode (either in object code or in source code). Moreover, the program may be provided on any computer-usable medium; the medium can be any element suitable to contain, store, communicate, propagate, or transfer the program. Examples of such medium are fixed disks (where the program can be pre-loaded), removable disks, tapes, cards, wires, fibres, wireless connections, networks, broadcast waves, and the like; for example, the medium may be of the electronic, magnetic, optical, electromagnetic, infrared, or semiconductor type.

In any case, the solution according to the present disclosure lends itself to be carried out with a hardware structure (for example, integrated in a chip of semiconductor material), or with a combination of software and hardware.

1. A method, for generating an aerosol cloud containing a high fine particle dose of a medicament with a device including a pressurized meter dose inhaler (MDI) reservoir containing a solution formulation of a medicament operated with HFA (hydrofluoroalkane) propellants, the MDI reservoir being connected to an electronic valve, the electronic valve being configured for receiving control signals from a microprocessor, the method including the steps of:
maintaining in a storage memory at least one set of medicament parameters, each set of parameters including a measure indicative of a total amount of aerosol medicament to be dispensed during a medicament session; and
controlling by the microprocessor the opening of the electronic valve, allowing the total amount of aerosol medicament being dispensed during a total inhalation time with a plurality of successive low volume pulses, the time interval between successive low volume pulses being less than 100 ms and the volume of medicament delivered during each single pulse being less than 5 μl so that the total inhalation time is minimized, while allowing the predetermined total amount of aerosol medicament being delivered.

2. The method of claim 1, wherein a duration of each low volume pulse is determined so that the fine particle fraction (FPF) of the aerosol medicament is maximized and the amount of FPF of aerosol delivered during each single pulse is calculated according to the following formula:
$$FPF ( \% ) = \frac{k}{{V}_{{P}^{0.25}}} ,$$
wherein k is function of HFA content and valve characteristics.

3. The method of claim 1, wherein the time interval between successive low volume pulses is determined so as to maximize fine particle fraction in atomizing a high volume formulation.

4. The method of claim 1, wherein the time interval between successive low volume pulses is 50 ms and the volume of medicament delivered during each single pulse is 2 μl.

5. The method of claim 1, wherein the storage memory includes a plurality of sets of medicament parameters and the calculation of the time interval between the plurality of successive low volume pulses and the amount of medicament delivered during each single pulse is performed responsive to a user selection of one of the plurality of sets of medicament parameters.

6. The method of claim 1, wherein the HFA propellants include one or more of the following: HFA 134a (1,1,1,2-tetrafluoroethane) or HFA 227 (1,1,1,2,3,3,3-heptafluoroproane).

7. The method of claim 1 wherein:
the MDI is connected to a plurality of electronic valve and to a plurality of reservoirs,
each reservoir is being coupled to at least one of the plurality of electronic valve, and
each valve is being adapted to deliver a different aerosol component.

8. A device for dispensing an aerosol medicament comprising:
a pressurized meter dose inhaler (MDI) operated with HFA (hydrofluoroalkane) propellants;
at least one reservoir adapted to contain aerosol medicaments;
at least one electronic valve, being connected to the MDI; and
a microprocessor for controlling the opening of the electronic valve, allowing a predetermined amount of aerosol medicament being dispensed during a total inhalation time with a plurality of successive low volume pulses the time interval between successive low volume pulses being less than 100 ms and the volume of medicament delivered during each single pulse being less than 5 μl so that the total inhalation time is minimized, while allowing the predetermined total amount of aerosol medicament being delivered.

9. The device of claim 8, wherein the electronic valve is connected to the MDI by means of a flexible conduct.

10. The device of claim 8 further comprising a plurality of electronic valves and a plurality of reservoirs, each reservoir being coupled to at least one of the plurality of electronic valves, each valve being adapted to deliver a different aerosol formulation.

11. The device of claim 10, wherein the plurality of electronic valves includes two electronic valves, each valve being adapted to deliver a different aerosol formulation.

12. A non-transitory storage device comprising a computer program including instructions for carrying out the method of claim 1, said instructions being configured to be is executed on a computer system.
